(19) United States
(12) Patent Application Publication
(10) Pub. No.: US 2013/0330317 A1
(43) Pub. Date: Dec. 12, 2013

(54) Methods for Treating Lysosomal Acid Lipase Deficiency

(71) Applicants: Synageva BioPharma Corp., Lexington, MA (US); Children's Hospital Medical Center, Cincinnati, OH (US)

(72) Inventors: Gregory Grabowski, Cincinnati, OH (US); Hong Du, Cincinnati, OH (US); Michael Heartlein, Boxbourugh, MA (US); Michael Concino, Bolton, MA (US); Paolo Martini, Boston, MA (US); Muthuraman Meiyappan, Jamaica Plain, MA (US); Alla Romashko, Lexington, MA (US); Brian Pescatore, Beverly, MS (US); Lawrence Charnas, Natick, MA (US)

(21) Appl. No.: 13/967,446

(22) Filed: Aug. 15, 2013

Related U.S. Application Data
(63) Continuation of application No. PCT/US2012/025233, filed on Feb. 15, 2012.

(51) Int. Cl. A61K 38/46

(52) U.S. Cl. CPC A61K 38/465
USPC 424/94.6

(57) ABSTRACT

The present invention provides compositions and methods for effective treatment of a lysosomal acid lipase deficiency (LALD) disease, in particular, Wolman's disease and Cholesteryl Ester Storage Disease (CESD). Among other things, the present invention provides a method of treating a lysosomal acid lipase deficiency (LALD) disease, including administering to an individual suffering from or susceptible to the LALD disease a therapeutic effective amount of a lysosomal acid lipase periodically at an administration interval such that lipid level in liver, spleen and/or small intestine is reduced by at least 20% as compared to an untreated control.

This application claims priority to U.S. Provisional Application Ser. No. 61/443,079, filed Feb. 15, 2011.

### BACKGROUND

Lysosomal acid lipase (LAL) deficiency is a rare but serious disease. Under normal conditions, the human body produces lysosomal acid lipase (LAL), an enzyme that breaks down fatty material (cholesteryl esters, triglycerides, di- and mono-acylglycerols). LAL Deficiency occurs when the body is not producing enough LAL. The lack of the LAL enzyme typically results in a massive build-up of fatty material in various tissues including liver, spleen, gut, blood vessel walls, and other important organs. As a result, LAL deficiency is typically associated with significant morbidity and mortality, and can affect individuals from infancy through adulthood.

Extremely low levels of the LAL enzyme typically causes early onset of LAL Deficiency, sometimes called Wolman Disease (also known as Wolman's disease or Wolman's syndrome). Early onset LAL Deficiency typically affects infants in the first year of life. For example, the build-up of fatty material in the cells of the gut prevents the body from absorbing nutrients. Consequently, Wolman disease is a rapidly progressive and typically fatal condition characterized by malabsorption, growth failure, and significant weight loss. These infants typically die during their first year of life from a failure to grow and from other complications due to liver failure.

Later onset LAL Deficiency is sometimes called Cholesteryl Ester Storage Disease (CESD) and can affect children and adults. Typically, CESD patients experience enlarged liver (hepatomegaly), cirrhosis, chronic liver failure, severe premature atherosclerosis, hardening of the arteries, or elevated levels of serum Low Density Lipoprotein (LDL). Children may also have calcium deposits in the adrenal glands and develop jaundice.

Currently, there is no approved therapy for LAL deficiency.

### SUMMARY OF THE INVENTION

The present invention provides compositions and methods for effective treatment of a lysosomal acid lipase deficiency (LALD) disease, in particular, Wolman's disease and Cholesteryl Ester Storage Disease (CESD). The present invention is, in part, based on the discovery that administration of a recombinant lysosomal acid lipase to an animal disease model is surprisingly effective in treating (e.g., ameliorating, inhibiting, or delaying onset of) various symptoms of LALD diseases, including massive accumulation of fatty materials in various organs (e.g., liver, spleen, gut), even at low doses.

In one aspect, the present invention provides methods of treating a lysosomal acid lipase deficiency (LALD) disease, including administering to an individual suffering from or susceptible to the LALD disease, a therapeutically effective amount of a lysosomal acid lipase periodically at an administration interval such that lipid level in liver, spleen and/or small intestine is reduced by at least about 20% as compared to an untreated control.

In some embodiments, the lysosomal acid lipase is recombinantly produced. In some embodiments, the lysosomal acid lipase is recombinantly produced from mammalian cells. In some embodiments, the lysosomal acid lipase is recombinantly produced from human cells.

In some embodiments, the lysosomal acid lipase has a half-life of about 5 hours or longer in the liver.

In some embodiments, the lipid level in liver, spleen and/or small intestine is reduced by at least about 40% as compared to an untreated control. In some embodiments, the lipid level in liver, spleen and/or small intestine is reduced by at least about 60% as compared to an untreated control. In some embodiments, the lipid level in liver, spleen and/or small intestine is reduced by at least about 80% as compared to an untreated control.

In some embodiments, the lipid level in the liver is reduced. In some embodiments, the lipid level in the spleen is reduced. In some embodiments, the lipid level in the small intestine is reduced.

In certain embodiments, the lipid level comprises cholesterol and/or triglycerides level.

In some embodiments, the untreated control is the lipid level in the individual being treated before the treatment. In some embodiments, the untreated control is the average lipid level in one or more control individuals suffering from the same form of the LALD disease without treatment.

In certain embodiments, administering the therapeutic effective amount of the lysosomal acid lipase further results in reducing liver and/or spleen weight. In some embodiments, the liver weight is reduced by more than about 20% as compared to an untreated control. In some embodiments, the spleen weight is reduced by more than about 30% as compared to an untreated control.

In some embodiments, the therapeutic effective amount may be at least about 0.05 mg/kg body weight. In some embodiments, the therapeutic effective amount may be at least about 0.1 mg/kg body weight. In some embodiments, the therapeutic effective amount may be at least about 0.5 mg/kg body weight. In some embodiments, the therapeutic effective amount may range from about 0.05-20 mg/kg body weight. In some embodiments, the therapeutic effective amount may range from about 0.05-1 mg/kg body weight.

It will be appreciated that the lysosomal acid lipase may be administered by any appropriate route. In some embodiments, the lysosomal acid lipase is administered intravenously. In some embodiments, the lysosomal acid lipase is administered intramuscularly. In some embodiments, the lysosomal acid lipase is administered intrathecally or intraventricularly.

It will be appreciated that the lysosomal acid lipase may be administered at any appropriate interval. For example, in some embodiments, the lysosomal acid lipase is administered monthly. In some embodiments, the lysosomal acid lipase is administered bimonthly. In some embodiments, the lysosomal acid lipase is administered weekly. In some embodiments, the lysosomal acid lipase is administered twice a week. In some embodiments, the lysosomal acid lipase is administered daily. In some embodiments, the lysosomal acid lipase is administered at an interval that is varied over time.

In some embodiments, the LALD disease is Wolman's disease. In some embodiments, the LALD disease is cholesteryl ester storage disease (CESD).

In certain embodiments, the lysosomal acid lipase has an amino acid sequence at least about 80% identical to human lysosomal acid lipase (SEQ ID NO:1). In some embodiments, the lysosomal acid lipase has an amino acid sequence at least about 90% identical to human lysosomal acid lipase (SEQ ID NO:1). In some embodiments, the lysosomal acid lipase has an amino acid sequence at least about 95% identical to human lysosomal acid lipase (SEQ ID NO:1). In some embodiments, the lysosomal acid lipase is human lysosomal acid lipase (SEQ ID NO:1).

In another aspect, the present invention provides methods of treating a lysosomal acid lipase deficiency (LALD) disease, including administering to an individual suffering from or susceptible to the LALD disease a therapeutically effective amount of a lysosomal acid lipase periodically at an administration interval as described in various embodiments above.

In yet another aspect, the present invention provides pharmaceutical compositions for treating a lysosomal acid lipase deficiency (LALD) disease, including a therapeutic effective amount of a lysosomal acid lipase as described in various embodiments above and a pharmaceutical carrier.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIG. 1 depicts an exemplary graph of a study design.

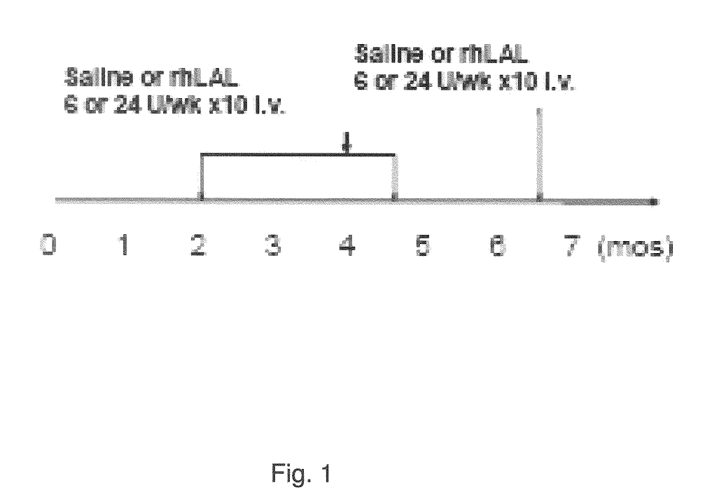

FIG. 2 depicts exemplary SDS-PAGE analysis of rhLAL. rhLAL was analyzed by SDS-PAGE and visualized by Coomassie stain and compared to BSA. Lanes 1-3: BSA, 1, 2.5, and 5 μg, respectively. Lanes 4-6: rhLAL, 1, 2.5 and 5 μg, respectively. Protein molecular weight markers are labeled on the left.

FIG. 3 depicts exemplary deglycosylation by PNGase. rhLAL was incubated with (lanes 1 and 4) or without PNGase F (lanes 2 and 3) for 1 hour (lanes 1 and 2) or overnight (lanes 3 and 4).

FIG. 4 depicts an exemplary time course of serum and tissue rhLAL activities in lal^{-/-} mice injected with three different doses of rhLAL. Plasma and tissue LAL activity was measured in serum samples collected after a single dose (24 U, 48 U, and 96 U per mouse) intravenous injection of rhLAL in lal^{-/-} mice. LAL activity is expressed as a percentage of injected.

FIG. 5 depicts an exemplary time course of liver rhLAL activities in lal^{-/-} mice injected with a single dose (48 U per mouse) intravenous injection of rhLAL. Activities are expressed as a percentage of injected. Inset depicts LAL activity in liver of lal^{-/-} mice expressed as a percentage of wild-type hepatic LAL activity. Data are mean±SE, n=5 mice.

FIG. 6 depicts an exemplary time course of hepatic rhLAL activities in lal^{-/-} mice injected with three different doses of rhLAL (24 U, 48 U, and 96 U per mouse). LAL activity is expressed as U/liver (top left panel), and percentage of injected (lower left panel). Exemplary hepatic disappearance of rhLAL is expressed in U/liver (right panel).

FIG. 7 depicts an exemplary time course of spleen rhLAL activities in lal^{-/-} mice injected with 48 U rhLAL. LAL activity is expressed as percentage of injected. n=5.

FIG. 8 depicts an exemplary cellular targeting of rhLAL in the liver and spleen. Paraffin embedded sections of liver and spleen from saline or rhLAL injected lal^{-/-} mice were processed by immunohistochemical staining with anti-hLAL antibody. Positive signals were evident in the Kupffer cells and in splenic macrophages of a representative section from an lal^{-/-} mouse (arrows). Original magnification: 400× for liver and 200× for spleen.

FIG. 9 depicts exemplary hepatic cellular localization of rhLAL. Paraffin embedded sections of liver from rhLAL injected (24 U, 48 U, and 96 U) lal^{-/-} mice were processed by immunohistochemical staining with anti-hLAL antibody at 40 minutes, 120 minutes, and 240 minutes post-injection.

FIG. 10 depicts exemplary kidney and small intestine cellular localization in macrophages of rhLAL. Paraffin embedded sections of kidney and small intestine from rhLAL injected lal^{-/-} mice were processed by immunohistochemical staining with anti-hLAL antibody and anti-Mac3 antibody.

FIG. 11 depicts exemplary correction of gross appearance and reduction of hepatosplenomegaly by rhLAL in mice. Gross views showing the yellow fatty liver, spleen and mesenteric lymph size comparison of lal^{-/-} mice at 4.5 and 6.5 months of age.

FIG. 12 depicts exemplary comparison of liver and spleen weight in wild-type mice (WT) and saline control (Saline) or rhLAL-treated (hLAL 6 U and hLAL 24 U) lal^{-/-} mice at 4.5 months and 6.5 months of age. Data represents mean±SE, n=5 mice.

FIG. 13 depicts exemplary correction of gross appearance and reduction of hepatosplenomegaly by rhLAL in lal^{-/-} mice. Gross views showing the yellow fatty liver, spleen and mesenteric lymph size comparison of lal^{-/-} mice treated with saline or rhLAL (6 U and 24 U) at 6.5 months of age. Liver and spleen weight in wild-type, untreated mice, saline control or rhLAL-treated (hLAL 6 U and hLAL 24 U) mice after weekly intravenous injection from 4 months to 6.5 months of age were determined.

FIG. 14 depicts exemplary correction of gross appearance and reduction of hepatosplenomegaly by rhLAL in lal^{-/-} mice treated with saline or 24 U rhLAL. Liver and spleen weight in saline control (Saline) or rhLAL-treated (hLAL 24 U and hLAL 72 U) lal^{-/-} mice after 3 doses were determined.

FIG. 15 depicts exemplary regression and/or prevention of progressive splenomegaly by rhLAL in lal^{-/-} mice. Spleen weight in wild-type, untreated lal^{-/-} mice, saline control or rhLAL-treated (hLAL 6 U and hLAL 24 U) lal^{-/-} mice after weekly intravenous injection from 2 months to 4.5 months of age or from 4 months to 6.5 months of age were determined. Spleen weight is expressed as a percentage of total body weight.

FIG. 16 depicts exemplary hematoxylin and eosin (H & E) staining of liver, spleen, and small intestine of wild-type and saline- or rhLAL-treated lal^{-/-} mice. Mice were 1.5, 2.5 or 4.5 months of age. Correction of lipid storage by rhLAL 6 U observed in Kupffer cells (panel E) is similar to a 2.5 month liver section (panel C). Correction of lipid storage by rhLAL 24 U observed in Kupffer cells (panel F) is similar to a 1.5 month liver section (panel B). Original magnification: 200×.

FIG. 17 depicts exemplary reduction of tissue neutral lipid in rhLAL-injected mice by Oil red-O staining.

FIG. 18 depicts exemplary reduction of tissue neutral lipid in liver of wild-type mice and saline or rhLAL-injected (6 U and 24 U) mice by Oil red-O staining Mice were intravenously injected weekly from 2 months of age to 4.5 months of age.

FIG. 19 depicts exemplary reduction of tissue neutral lipid in small intestine of wild-type mice, untreated lal^{-/-} mice (at 1.5 months and 2.5 months of age) and saline or rhLAL-injected (6 U and 24 U) lal^{-/-} mice by Oil red-O staining. Mice were intravenously injected weekly from 2 months of age to 4.5 months of age (2.5 months of injection).

FIG. 20 depicts exemplary reduction of tissue neutral lipid in liver of wild-type mice, untreated lal^{-/-} mice (at 2.5 months and 4.5 months of age) and saline or rhLAL-injected (6 U and 24 U) lal^{-/-} mice by Oil red-O staining. Mice were intravenously injected weekly from 4 months of age to 6.5 months of age.

FIG. 21 depicts exemplary reduction of tissue neutral lipid in small intestine of wild-type mice, untreated lal^{-/-} mice (at 2.5 months and 4.5 months of age) and saline or rhLAL-injected (6 U and 24 U) lal^{-/-} mice by Oil red-O staining. Mice were intravenously injected weekly from 4 months of age to 6.5 months of age.

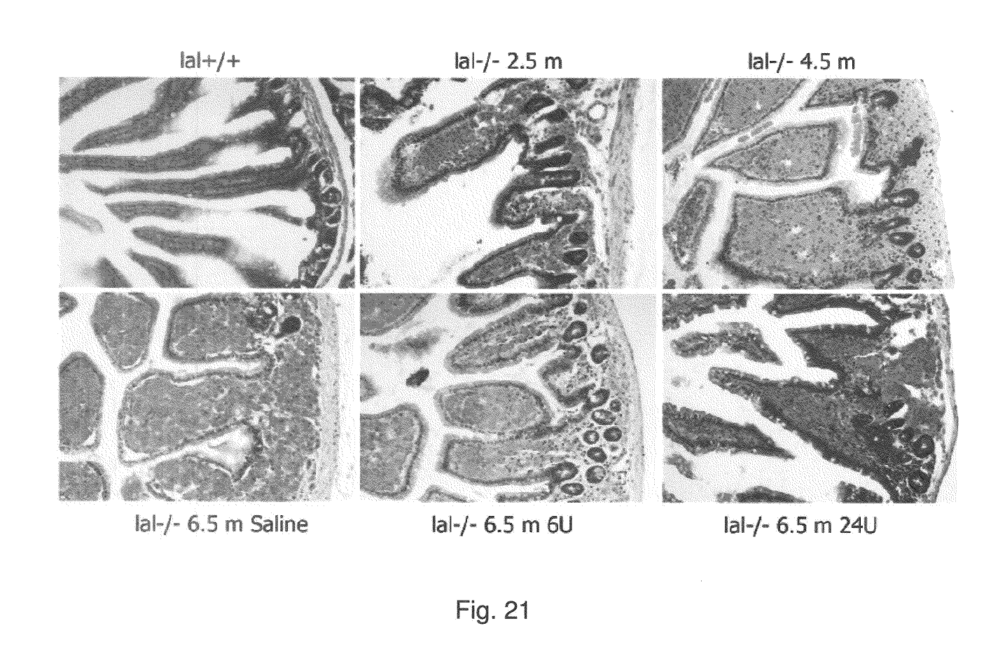

FIG. 22 depicts exemplary reduction of tissue neutral lipid in liver, spleen, and small intestine of saline or rhLAL-injected (24 U and 72 U) mice by Oil red-O staining.

FIG. 23 depicts exemplary reduction of tissue neutral lipid in rhLAL-injected mice by tissue lipid analyses. Cholesterol and triglycerides were measured in liver, spleen, intestine, and lymph node of wild-type and saline or rhLAL-treated (hLAL 6 U and hLAL 24 U) lal^{-/-} mice at 4.5 months and 6.5 months of age.

FIG. 24 depicts an exemplary graph of a study design.

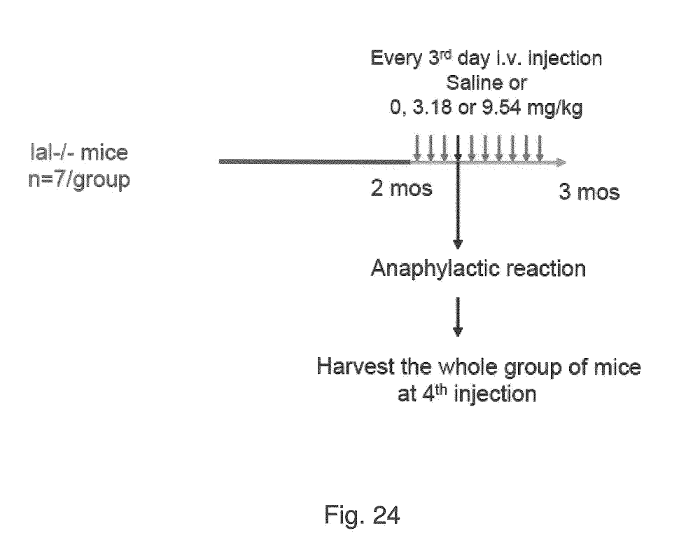

FIG. 25 depicts an exemplary graph of a study design for rhLAL treatment of “young mice” and “older mice”.

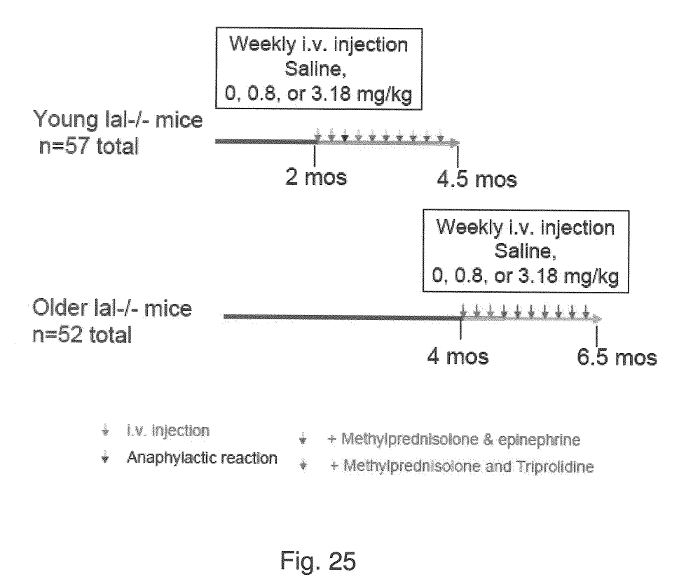

### DEFINITIONS

In order for the present invention to be more readily understood, certain terms are first defined below. Additional definitions for the following terms and other terms are set forth throughout the specification.

Amelioration: As used herein, the term “amelioration” is meant the prevention, reduction or palliation of a state, or improvement of the state of a subject. Amelioration includes, but does not require complete recovery or complete prevention of a disease condition. In some embodiments, amelioration includes reduction of accumulated materials inside lysosomes of relevant diseases tissues.

Biologically active: As used herein, the phrase “biologically active” refers to a characteristic of any agent that has activity in a biological system, and particularly in an organism. For instance, an agent that when administered to an organism has a biological effect on that organism is considered to be biologically active. In particular embodiments, where a protein or polypeptide is biologically active, a portion of that protein or polypeptide that shares at least one biological activity of the protein or polypeptide is typically referred to as a “biologically active” portion.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of lysosomal storage disease (e.g., LAL deficiency) as the individual being treated, who is about the same age as the individual being treated (to ensure that the stages of the disease in the treated individual and the control individual(s) are comparable).

Individual, subject, patient: As used herein, the terms “subject,” “individual” or “patient” refer to a human or a non-human mammalian subject. The individual (also referred to as “patient” or “subject”) being treated is an individual (fetus, infant, child, adolescent, or adult human) suffering from a lysosomal storage disease, for example, LAL deficiency disease (e.g., early-onset such as Wolman's disease; later-onset such as Cholesteryl Ester Storage Disease (CESD)).

Lysosomal enzyme deficiency: As used herein, “lysosomal enzyme deficiency” refers to a group of genetic disorders that result from deficiency in at least one of the enzymes (e.g., lysosomal acid lipase) that are required to break macromolecules (e.g., fatty materials) down to peptides, amino acids, monosaccharides, nucleic acids and fatty acids in lysosomes. As a result, individuals suffering from lysosomal enzyme deficiencies have accumulated materials in various tissues (e.g., liver, spleen, gut, blood vessel walls and other organs).

Lysosomal enzyme: As used herein, the term “lysosomal enzyme” refers to any enzyme (e.g., lysosomal acid lipase (LAL)) that is capable of reducing accumulated materials in mammalian tissues or that can rescue or ameliorate one or more lysosomal enzyme deficiency symptoms (e.g., developmental impairment, liver failure, etc.). Lysosomal enzymes suitable for the invention include both wild-type or modified lysosomal enzymes and can be produced using recombinant and synthetic methods or purified from nature sources.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues will appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains Substitution of one amino acid for another of the same type may often be considered a “homologous” substitution.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Therapeutically effective amount: As used herein, the term “therapeutically effective amount” refers to an amount of a therapeutic lysosomal enzyme (e.g., lysosomal acid lipase (LAL)) protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Treatment: As used herein, the term “treatment” (also “treat” or “treating”) refers to any administration of a therapeutic protein (e.g., lysosomal acid lipase deficiency (LALD) disease) that partially or completely alleviates, ameliorates, relieves, inhibits, delays onset of, reduces severity of and/or reduces incidence of one or more symptoms or features of a particular disease, disorder, and/or condition (e.g., lysosomal acid lipase deficiency (LALD) disease). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### DETAILED DESCRIPTION

The present invention provides compositions and methods for treatment of lysosomal acid lipase deficiency (LALD) diseases, in particular, Wolman's disease and/or CESD. Among other things, the present invention provides compositions and methods for administering to a mammal suffering from or susceptible to a LALD disease a therapeutic effective amount of a lysosomal acid lipase.

Various aspects of the invention are described in detail in the following sections. The use of sections is not meant to limit the invention. Each section can apply to any aspect of the invention. In this application, the use of “or” means “and/or” unless stated otherwise.

### Lysosomal Acid Lipase Deficiency

Lysosomal Acid Lipase (LAL) is an enzyme that hydrolyzes cholesterol esters and triglycerides in the lysosome. LAL deficiency disease (LALD) is an autosomal recessive genetic disorder that results from a lack of LAL activity and typically leads to massive build-up of fatty material in various tissues. LALD can manifest as early onset of LALD, sometimes called Wolman Disease (also known as Wolman's disease, Wolman's syndrome), which typically affects infants in the first year of life. Alternatively or additionally, LALD can manifest as later onset LALD (sometimes called Cholesteryl Ester Storage Disease (CESD)), which can affect children and adults.

Individuals affected by Wolman Disease typically have harmful amounts of lipids that accumulate in the spleen, liver, bone marrow, small intestine, adrenal glands, lymph nodes, among other tissues. In some cases, calcium deposits are seen in the adrenal glands of affected individuals. Infants with Wolman disease usually appear healthy at birth, but soon develop signs and symptoms of the disorder, including hepatosplenomegaly, developmental impairment or cachexia (poor weight gain, weight loss, tissue weakness or wasting), low muscle tone, jaundice, vomiting, diarrhea, developmental delay, anemia, and/or poor absorption of nutrients from food (see, for example, Genetics Home Reference http://ghr.nlm.nih.gov/, the entire contents of which is incorporated herein by reference). These infants typically develop severe malnutrition and die during their first year of life. For example, Crocker and colleagues presented case studies of three individuals affected by Wolman Disease (Crocker, et al. Pediatrics “Wolman's Disease: Three New Patients with a Recently Described Lipidosis”; 1965, the entire contents of which is incorporated herein by reference). Each of the three individuals studied by Crocker and colleagues were admitted to the hospital due at least in part to failure to gain or difficulty in weight gain. The individuals also suffered from diarrhea and vomiting, among other things, and despite nutritional efforts, each of the infants died in a condition of inanition.

Later onset LAL Deficiency is sometimes called Cholesteryl Ester Storage Disease (CESD) and can affect children and adults. Typically, CESD patients experience enlarged liver (hepatomegaly), cirrhosis, chronic liver failure, severe premature atherosclerosis, hardening of the arteries, or elevated levels of serum Low Density Lipoprotein (LDL). Children may also have calcium deposits in the adrenal glands and develop jaundice.

Non-human animal models have been developed to study LALD. For example, a mouse model with a LAL null mutation (lal−/−) was produced by targeted disruption of the mouse gene (see, for example, Du et al. Human Molecular Genetics 7(9):1347 1998, the contents of which are incorporated herein by reference). Du and colleagues have characterized LAL knockout mice and demonstrated that homozygous lal−/− mice produce no LAL mRNA or protein and demonstrate no enzyme activity. The LAL knockout mouse model (lal−/−) resembles human LALD with storage of cholesteryl esters and triglycerides in multiple organs, loss of subcutaneous and omental fat, and hepatosplenomegaly.

### Lysosomal Acid Lipase

A lysosomal acid lipase enzyme suitable for the present invention includes any enzyme that is capable of reducing accumulated fatty materials in mammalian tissues or that can rescue or ameliorate one or more lysosomal acid lipase deficiency (LALD) disease symptoms (e.g., developmental impairment, or liver failure, etc.).

In some embodiments, human lysosomal acid lipase (LAL) (also referred to as human lysosomal acid lipid lipase or cholesteryl ester hydrolase) is used. Typically, a mature form of human LAL is used. The sequence of a mature human LAL (SEQ ID NO:1) is listed below in Table 1 and described below. Typically, human LAL is first synthesized as a precursor protein containing a 21-amino acid signal peptide at the N-terminus. The signal peptide is cleaved post-translationally resulting in the mature form of human LAL. The sequences of the signal peptide (SEQ ID NO:2) and the full length precursor protein (SEQ ID NO:3) are also shown in Table 1.

TABLE 1
Human Lysosomal Acid Lipase/Cholesteryl Ester
Hydrolase (P38571)

| | |
|---|---|
| Mature form | SGGKLTAVDPETNMNVSEIISYWGFPSEEYLVETEDGYILCLNRIPHGRKNHSDKGP |
| | KPVVFLQHGLLADSSNWVTNLANSSLGFILADAGFDVWMGNSRGNTWSRKHKTLSVS |
| | QDEFWAFSYDEMAKYDLPASINFILNKTGQEQVYYVGHSQGTTIGFIAFSQIPELAK |
| | RIKMFFALGPVASVAFCTSPMAKLGRLPDHLIKDLFGDKEFLPQSAFLKWLGTHVCT |
| | HVILKELCGNLCFLLCGFNERNLNMSRVDVYTTHSPAGTSVQNMLHWSQAVKFQKFQ |
| | AFDWGSSAKNYFHYNQSYPPTYNVKDMLVPTAVWSGGHDWLADVYDVNILLTQITNL |
| | VFHESIPEWEHLDFIWGLDAPWRLYNKIINLMRKYQ (SEQ ID NO: 1) |
| Signal | MKMRFLGLVVCLVLWTLHSEG (SEQ ID NO: 2) |
| Sequence | |
| Full Length | MKMRFLGLVVCLVLWTLHSEGSGGKLTAVDPETNMNVSEIISYWGFPSEEYLVETE |
| Precursor | DGYILCLNRIPHGRKNHSDKGPKPVVFLQHGLLADSSNWVTNLANSSLGFILADAG |
| | FDVWMGNSRGNTWSRKHKTLSVSQDEFWAFSYDEMAKYDLPASINFILNKTGQEQV |
| | YYVGHSQGTTIGFIAFSQIPELAKRIKMFFALGPVASVAFCTSPMAKLGRLPDHLI |
| | KDLFGDKEFLPQSAFLKWLGTHVCTHVILKELCGNLCFLLCGFNERNLNMSRVDVY |
| | TTHSPAGTSVQNMLHWSQAVKFQKFQAFDWGSSAKNYFHYNQSYPPTYNVKDMLVPT |
| | AVWSGGHDWLADVYDVNILLTQITNLVFHESIPEWEHLDFIWGLDAPWRLYNKIINL |
| | MRKYQ (SEQ ID NO: 3) |

Natural variants of human lysosomal acid lipase polypeptides are known. For example, in some embodiments, residues 1-56 of SEQ ID NO:3, corresponding to residues 1-35 of SEQ ID NO:1, are deleted. In some embodiments, residues 57-76 of SEQ ID NO:3 (DGYILCLNRIPHGRKNHSDK), corresponding to residues 36-55 of SEQ ID NO:1, are replaced with MACLEFVPFDVQMCLEFLPS (SEQ ID NO:4). In some embodiments, residue 16 of SEQ ID NO:3 has a Thr to Pro substitution. In some embodiments, residue 23 of SEQ ID NO:3, corresponding to residue 2 of SEQ ID NO:1, has a Gly to Arg substitution. In some embodiments, residue 29 of SEQ ID NO:3, corresponding to residue 8 of SEQ ID NO:1, has a Val to Leu substitution. In some embodiments, residue 228 of SEQ ID NO:3, corresponding to residue 207 of SEQ ID NO:1, has a Phe to Ser substitution.

In some embodiments, a lysosomal acid lipase enzyme suitable for the present invention is substantially homologous to SEQ ID NO:1. In some embodiments, a suitable lysosomal acid lipase enzyme has a sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:1

In some embodiments, a lysosomal acid lipase enzyme suitable for the present invention is substantially identical to SEQ ID NO:1. In some embodiments, a suitable lysosomal acid lipase enzyme has a sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:1.

In some embodiments, a lysosomal acid lipase enzyme suitable for the present invention is a fragment of human LAL or a fusion protein containing a lysosomal acid lipase such as human LAL, or a portion thereof.

A lysosomal acid lipase suitable for the present invention may be produced by any available means. For example, lysosomal acid lipase enzymes may be recombinantly produced by utilizing a host cell system engineered to express an LAL polypeptide-encoding nucleic acid. Alternatively or additionally, lysosomal acid lipase enzymes may be partially or fully prepared by chemical synthesis. Alternatively or additionally, lysosomal acid lipase enzymes may also be purified from natural sources.

Where lysosomal acid lipase enzymes are recombinantly produced, any expression system can be used. To give but a few examples, known expression systems include, for example, egg, baculovirus, plant, yeast, or mammalian cells.

In some embodiments, lysosomal acid lipase enzymes suitable for the present invention are produced in mammalian cells. Non-limiting examples of mammalian cells that may be used in accordance with the present invention include BALB/c mouse myeloma line (NSO/l, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); human fibrosarcoma cell line (HT1080); baby hamster kidney cells (BHK, ATCC CCL 10); Chinese hamster ovary cells+/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68, 1982); MRC 5 cells; FS4 cells; and a human hepatoma line (Hep G2).

In some embodiments, lysosomal acid lipase enzymes suitable for the present invention are produced in avian expression systems, e.g. in eggs of chimeric chickens. Exemplary methodologies for expressing proteins, including lysosomal acid lipases, in avian expression systems are described in PCT Publication WO 2004/015123 and U.S. Pub. Nos. 20060191026, 20090178147; 20090180989; 20100083389; and 2010033219, the entire contents of each of which are incorporated herein by reference.

In some embodiments, lysosomal acid lipase enzymes suitable for the present invention are produced in plant expression systems, e.g. in tobacco plants or related species (e.g., Nicotiana species). In some embodiments, lysosomal acid lipase enzymes are expressed in Nicotiana benthamiana. Exemplary methodologies for expressing proteins, including lysosomal acid lipases, in plant expression systems are known in the art. For example, GENEWARE® technology utilizes a modified tobacco mosaic virus vector to express heterologous proteins within a tobacco plant. Expressed proteins may subsequently be isolated and/or purified.

In some embodiments, lysosomal acid lipase enzymes suitable for the present invention are produced in yeast expression systems, e.g. in methylotrophic yeast. In some embodiments, lysosomal acid lipase enzymes are expressed in Pichia pastoris. Exemplary methodologies for expressing proteins, including lysosomal acid lipases, in yeast expression systems are known in the art (see, for example, Daly, et al. J Mol. Recognit. 18:119 (2005), the contents of which is incorporated herein by reference).

It will be appreciated that other expression systems are known in the art and can be used to produce lysosomal acid lipase enzymes described herein.

In some embodiments, lysosomal acid lipase enzymes produced by a suitable expression system may have similar or identical glycosylation level or pattern to that of a naturally-occurring human LAL. In some embodiments, lysosomal acid lipase enzymes produced by a suitable expression system may have increased or decreased glycosylation level or altered glycosylation pattern as compared to a naturally-occurring human LAL. For example, lysosomal acid lipases may be produced using transgene-augmented glycosylation avians as described in U.S. Pub. No. 20090178147, the disclosure of which is incorporated herein by reference.

In some embodiments, suitable lysosomal acid lipase enzymes may be post-translationally modified to alter the glycosylation level or pattern of the enzyme. For example, a lysosomal acid lipase enzyme may be modified to increase or decrease glycosylation levels. In some embodiments, a lysosomal acid lipase enzyme may be de-glycosylated.

Typically, lysosomal acid lipase enzymes suitable for the present invention have desirable pharmacokinetics and pharmacodynamics. In some embodiments, suitable lysosomal acid lipase enzymes have a serum half-life longer than about 10 minutes, or about 20 minutes, or about 30 minutes, or about 40 minutes, or about 50 minutes, or about 1 hour. In some embodiments, suitable lysosomal acid lipase enzymes have a half-life longer than about 1 hour, or about 2 hours, or about 3 hours, or about 4 hours, or about 5 hours, or about 6 hours, or about 7 hours, or about 8 hours, or about 9 hours, or about 10 hours, or about 15 hours, or about 20 hours in the liver, spleen, or small intestine.

### Treatment of LAL Deficiency

Methods of the present invention may be used to effectively treat individuals suffering from or susceptible to LALD diseases, in particular, those individuals affected by Wolman's disease or cholesteryl ester storage disease (CESD). The terms, “treat” or “treatment,” as used herein, refers to amelioration of one or more symptoms associated with the disease, prevention or delay of the onset of one or more symptoms of the disease, and/or lessening of the severity or frequency of one or more symptoms of the disease.

In some embodiments, treatment refers to reduction of accumulation of fatty material (e.g., lipids such as cholesterol and/or triglycerides) in various tissues (e.g., liver, spleen, gut, blood vessel walls, bone marrow, adrenal glands (small hormone-producing glands on top of each kidney), and lymph nodes, etc.). In some embodiments, treatment results in a reduction of lipid accumulation by more than about 10%, more than about 20%, more than about 30%, more than about 40%, more than about 50%, more than about 60%, more than about 70%, more than about 80%, more than about 90%, or more than about 95%. In some embodiments, treatment results in substantial elimination of lipid accumulation in various tissues (e.g., liver, spleen, gut, blood vessel walls, bone marrow, adrenal glands (small hormone-producing glands on top of each kidney), and lymph nodes, etc.).

In some embodiments, treatment refers to improved gross tissue pathology in various tissues (e.g., liver, spleen, small intestines). For example, treatment can result in decreased relative tissue (e.g., liver, spleen, small intestine) weight and/or volume to total body weight. In some embodiments, treatment refers to reduced relative tissue (e.g., liver, spleen, small intestine) weight and/or volume to total body by at least about 10%, about 20%, about 30%, about 40%, about 50%, about 60%, about 70%, about 80%, about 90%, or about 95% as compared to the pre-treatment state. In some embodiments, treatment according to the present invention results in a relative tissue (e.g., liver, spleen, small intestine) weight and/or volume similar to that of an healthy individual at the same developmental stage (e.g., same age and gender).

It will be appreciated that tissue analyses, e.g., fat content and/or gross tissue pathology, and/or relative tissue weight or volume may be determined by any appropriate method available in the art and/or described herein. For example, in some embodiments, tissue pathology and relative weight/volume is analyzed by magnetic resonance imaging (MRI) and/or magnetic resonance spectroscopy (MRS). E.g., see d'Assignies et al. Magnetic Resonance 21:301 2011, the contents of which are incorporated herein by reference. d'Assignies and colleagues demonstrated simultaneous assessment of liver volume and whole liver fat content in patients by MRI/MRS. Additional tissue analysis methods include, but are not limited to, computed tomography (CT), tissue biopsy, biochemical tests of tissue function, ultrasound, Xenon clearance rates, or combinations thereof. Thus, in some embodiments, treatment refers to improved gross tissue pathology, morphology, relative tissue weight or volume, or fat content as determined by one or more methods described herein or known in the art.

In some embodiments, treatment refers to reduction of fatty material (e.g., lipids such as cholesterol and/or triglycerides) in serum. In some embodiments, treatment results in a reduction of lipid in serum by more than about 10%, more than about 20%, more than about 30%, more than about 40%, more than about 50%, more than about 60%, more than about 70%, more than about 80%, more than about 90%, more than about 95%, or more, as compared to the pre-treatment level. In some embodiments, treatment according to the present invention results in a serum fatty material (e.g., lipids such as cholesterol and/or triglycerides) level similar to that of a healthy individual at the same developmental stage (e.g., same age and gender).

In some embodiments, treatment refers to improvement of tissue function (e.g., liver, heart, muscle, kidney, etc.) as determined by the presence and/or amounts or activities of certain enzymes in the blood. For example, tissue function may be measured by the presence and/or amounts of aspartate aminotransferase (AST) (also known as serum glutamic oxaloacetic transaminase (SGOT)) and/or alanine aminotransferase (ALT) (also known as serum glutamic pyruvic transaminase (SGPT)). AST is normally found in liver, heart, muscle, kidney, and brain, and is typically released into serum when any one of these tissue is damaged. ALT is normally found largely in the liver, although it can be found in other tissues in smaller amounts. ALT is typically released into the serum as a result of liver injury and serves as a fairly specific indicator of liver status. Other enzymes indicative of various tissue (e.g., liver, heart, muscle, kidney, etc.) function are known in the art and can be used to monitor the treatment efficacy according to the present invention.

The terms, “improve,” “increase” or “reduce,” as used herein, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of lysosomal acid lipase deficiency (LALD) disease (either early-onset (e.g., Wolman's disease) or later-onset (e.g., cholesteryl ester storage disease (CESD)) as the individual being treated, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having lysosomal acid lipase deficiency (LALD) disease (either early-onset (e.g., Wolman's disease) or later-onset (e.g., cholesteryl ester storage disease (CESD)) or having the potential to develop lysosomal acid lipase deficiency (LALD) disease. The individual can have residual endogenous lysosomal acid lipase (LAL) activity, or no measurable activity. For example, the individual having lysosomal acid lipase deficiency (LALD) disease can have LAL activity that is less than about 1% of normal LAL activity (i.e., LAL activity that is usually associated with early-onset Wolman's disease), or LAL activity that is about 1 to about 10% of normal LAL activity (i.e., LAL activity that is usually associated with later-onset cholesteryl ester storage disease (CESD)).

In some embodiments, the individual is an individual who has been recently diagnosed with the disease. Typically, early treatment (treatment commencing as soon as possible after diagnosis) is important to minimize the effects of the disease and to maximize the benefits of treatment.

### Administration of Lysosomal Acid Lipase

In the methods of the invention, the Lysosomal Acid Lipase (LAL) is typically administered to the individual alone, or in compositions or medicaments comprising Lysosomal Acid Lipase (LAL) (e.g., in the manufacture of a medicament for the treatment of the disease), as described herein. The compositions can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interference with their activity. In some embodiments, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration. The Lysosomal Acid Lipase (LAL) can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Lysosomal Acid Lipase (LAL) (or a composition or medicament containing Lysosomal Acid Lipase (LAL)) is administered by any appropriate route. In some embodiments, Lysosomal Acid Lipase (LAL) is administered intravenously. In some embodiments, Lysosomal Acid Lipase (LAL) is administered subcutaneously. In some embodiments, Lysosomal Acid Lipase (LAL) is administered by direct administration to a target tissue, such as heart or muscle (e.g., intramuscular), or nervous system (e.g., direct injection into the brain; intraventricularly; intrathecally). Alternatively, Lysosomal Acid Lipase (LAL) (or a composition or medicament containing Lysosomal Acid Lipase (LAL)) can be administered parenterally, transdermally, or transmucosally (e.g., orally or nasally). More than one route can be used concurrently, if desired.

Lysosomal Acid Lipase (LAL) (or composition or medicament containing Lysosomal Acid Lipase (LAL)) can be administered alone, or in conjunction with other agents, such as antihistamines (e.g., diphenhydramine) or immunosuppressants or other immunotherapeutic agents that counteract anti-Lysosomal Acid Lipase (LAL) antibodies. The term, “in conjunction with,” indicates that the agent is administered prior to, at about the same time as, or following the Lysosomal Acid Lipase (LAL) (or composition containing Lysosomal Acid Lipase (LAL)). For example, the agent can be mixed into a composition containing Lysosomal Acid Lipase (LAL), and thereby administered contemporaneously with the Lysosomal Acid Lipase (LAL); alternatively, the agent can be administered contemporaneously, without mixing (e.g., by “piggybacking” delivery of the agent on the intravenous line by which the Lysosomal Acid Lipase (LAL) is also administered, or vice versa). In another example, the agent can be administered separately (e.g., not admixed), but within a short time frame (e.g., within 24 hours) of administration of the Lysosomal Acid Lipase (LAL). In some embodiments, Lysosomal Acid Lipase (LAL) (or composition containing Lysosomal Acid Lipase (LAL)) is administered in conjunction with an immunosuppressive or immunotherapeutic regimen designed to reduce amounts of, or prevent production of, anti-Lysosomal Acid Lipase (LAL) antibodies. For example, a protocol similar to those used in hemophilia patients (Nilsson et al. (1988) N. Engl. J. Med., 318:947-50) can be used to reduce anti-Lysosomal Acid Lipase (LAL) antibodies. Such a regimen can be used in individuals who have, or are at risk of having, anti-Lysosomal Acid Lipase (LAL) antibodies. In some embodiments, the immunosuppressive or immunotherapeutic regimen is begun prior to the first administration of Lysosomal Acid Lipase (LAL), in order to minimize the possibility of production of anti-Lysosomal Acid Lipase (LAL) antibodies.

Lysosomal Acid Lipase (LAL) (or composition or medicament containing Lysosomal Acid Lipase (LAL)) is administered in a therapeutically effective amount (i.e., a dosage amount that, when administered at regular intervals, is sufficient to treat the disease, such as by ameliorating symptoms associated with the disease, preventing or delaying the onset of the disease, and/or also lessening the severity or frequency of symptoms of the disease, as described above). As used herein, the therapeutic effective amount is also referred to as therapeutic effective dose or therapeutic effective dosage amount. The dose which will be therapeutically effective for the treatment of the disease will depend on the nature and extent of the disease's effects, and can be determined by standard clinical techniques. In addition, in vitro or in vivo assays may optionally be employed to help identify optimal dosage ranges, such as those exemplified below. The precise dose to be employed will also depend on the route of administration, and the seriousness of the disease, and should be decided according to the judgment of a practitioner and each patient's circumstances. Effective doses may be extrapolated from dose-response curves derived from in vitro or animal model test systems (e.g., as described by the U.S. Department of Health and Human Services, Food and Drug Administration, and Center for Drug Evaluation and Research in “Guidance for Industry: Estimating Maximum Safe Starting Dose in Initial Clinical Trials for Therapeutics in Adult Healthy Volunteers”, Pharmacology and Toxicology, July 2005, the entire contents of which are incorporated herein by reference).

In some embodiments, the therapeutically effective amount can be, for example, more than about 0.01 mg/kg, more than about 0.05 mg/kg, more than about 0.1 mg/kg, more than about 0.5 mg/kg, more than about 1.0 mg/kg, more than about 1.5 mg/kg, more than about 2.0 mg/kg, more than about 2.5 mg/kg, more than about 5.0 mg/kg, more than about 7.5 mg/kg, more than about 10 mg/kg, more than about 12.5 mg/kg, more than about 15 mg/kg, more than about 17.5 mg/kg, more than about 20 mg/kg, more than about 22.5 mg/kg, or more than about 25 mg/kg body weight. In some embodiments, a therapeutically effective amount can be about 0.01-25 mg/kg, about 0.01-20 mg/kg, about 0.01-15 mg/kg, about 0.01-10 mg/kg, about 0.01-7.5 mg/kg, about 0.01-5 mg/kg, about 0.01-4 mg/kg, about 0.01-3 mg/kg, about 0.01-2 mg/kg, about 0.01-1.5 mg/kg, about 0.01-1.0 mg/kg, about 0.01-0.5 mg/kg, about 0.01-0.1 mg/kg, about 1-20 mg/kg, about 4-20 mg/kg, about 5-15 mg/kg, about 5-10 mg/kg body weight. In some embodiments, a therapeutically effective amount may be about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, body weight, or more. In some embodiments, the therapeutically effective amount may be no greater than about 30 mg/kg, no greater than about 20 mg/kg, no greater than about 15 mg/kg, no greater than about 10 mg/kg, no greater than about 7.5 mg/kg, no greater than about 5 mg/kg, no greater than about 4 mg/kg, no greater than about 3 mg/kg, no greater than about 2 mg/kg, or no greater than about 1 mg/kg body weight or less.

The effective dose for a particular individual can be varied (e.g., increased or decreased) over time, depending on the needs of the individual. For example, in times of physical illness or stress, or if anti-Lysosomal Acid Lipase (LAL) antibodies become present or increase, or if disease symptoms worsen, the dosage amount can be increased.

A therapeutically effective amount of Lysosomal Acid Lipase (LAL) (or composition or medicament containing Lysosomal Acid Lipase (LAL)) is administered at regular intervals, depending on the nature and extent of the disease's effects, and on an ongoing basis. Administration at an “interval,” as used herein, indicates that the therapeutically effective amount is administered periodically (as distinguished from a one-time dose). The interval can be determined by standard clinical techniques. In some embodiments, Lysosomal Acid Lipase (LAL) is administered bimonthly, monthly, twice monthly, triweekly, biweekly, weekly, twice weekly, thrice weekly, or daily. The administration interval for a single individual need not be a fixed interval, but can be varied over time, depending on the needs of the individual. For example, in times of physical illness or stress, if anti-Lysosomal Acid Lipase (LAL) antibodies become present or increase, or if disease symptoms worsen, the interval between doses can be decreased.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

The invention additionally pertains to a pharmaceutical composition comprising human Lysosomal Acid Lipase (LAL), as described herein, in a container (e.g., a vial, bottle, bag for intravenous administration, syringe, etc.) with a label containing instructions for administration of the composition for treatment of Lysosomal Acid Lipase Deficiency (LALD) disease (e.g., Wolman's disease or CESD), such as by the methods described herein.

The invention will be further and more specifically described by the following examples. Examples, however, are included for illustration purposes, not for limitation.

### EXAMPLES

### Example 1

### Production of Recombinant Human Lysosomal Acid Lipase (rhLAL)

Experiments described in this example show that recombinant LAL can be expressed and purified from mammalian cells.

Specifically, cultured human cells were transfected with a plasmid as provided above containing human Lysosomal Acid Lipase open reading frame by electroporation and cloned by limiting dilution. Stable clones were selected using cloning media containing 0.4 mg/mL G418. Clones were expanded and rhLAL expression and activity was analyzed using ELISA and LAL activity assays. Such methods are well known and within the skill of one of ordinary skill in the art.

In total, 384 clones were analyzed for rhLAL expression and activity. Clone 35 was determined to have the highest and most stable rhLAL expression. Expression in shake flasks was 3-4 pcd (picograms per cell per day) on average and expression in wave reactor was 4-6 pcd on average. Clone 35 was expanded and a cell bank was prepared.

Clone 35 was seeded into 5 mL Wave cultures in 10 L wave bags. Wave bags were perfused at 5 L per day and conditioned media (CM) were harvested, filtered and stored at 4° C. for up to 7 days, until concentration by ultra-filtration. CM was concentrated using 10 kDa MWCO UF/DF concentrator and stored at −20° C. for 10-40 days until processed. Prior to purification, concentrated CM was thawed and the conductivity was increased by the addition of NaCl to a final concentration of 200 mM. Purification involved butyl-Sepharose 4 FF column capture followed by a polishing step with Q Sepharose FF column. Purified rhLAL was dialyzed against the final storage buffer, PBS pH=6.5, sterile filtered and stored at −80° C.

Certain characteristics of the rhLAL protein were determined. The DNA coding sequence predicts the molecular weight to be 43 kDa. Purified rhLAL was subjected to denaturing SDS-PAGE Western and Coomassie, and native size exclusion chromatography. Post translational modifications of rhLAL were determined by glycodigestion followed by SDS-PAGE Western or native isoelectric focusing (IEF).

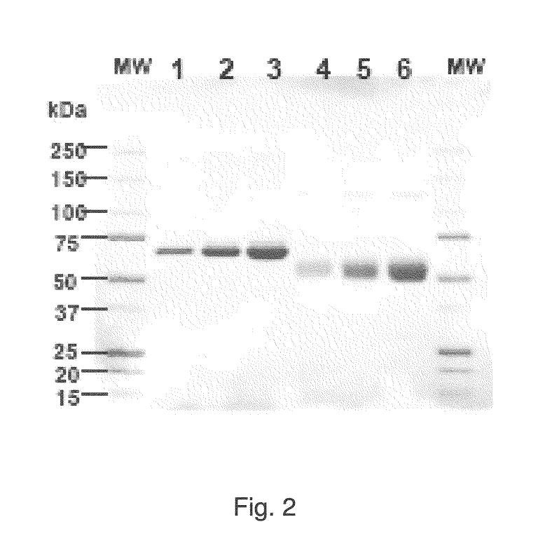

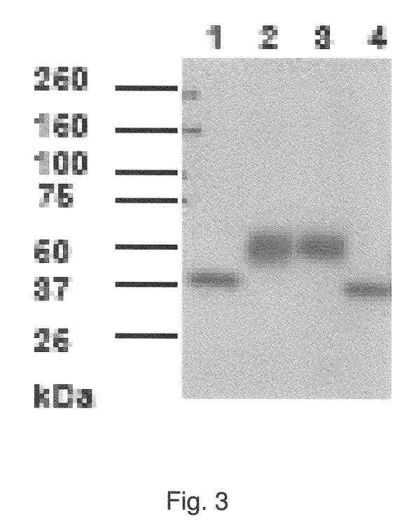

As shown in FIG. 2, rhLAL was analyzed by denaturing SDS-PAGE and visualized by Coomassie stain and compared to BSA. Lanes 1-3 contained 1, 2.5, and 5 μg of BSA, respectively. Lanes 4-6 contained 1, 2.5 and 5 μg of purified rhLAL, respectively. Protein molecular weight markers are labeled on the left. Purified rhLAL demonstrated an apparent molecular weight of ˜54.5 kDa. Human LAL protein has 6 potential N glycosylation sites based on its primary amino acid sequence. Deglycosylation of purified rhLAL by incubation with PNGase F led to a decrease in molecular weight of the protein to ˜42 kDa compared to untreated protein with a molecular weight of ˜50 kDa (FIG. 3). These studies indicated the presence of carbohydrates on the protein backbone of purified rhLAL.

### Example 2

### rhLAL Half-Life and Tissue Targeting In Vivo

The experiments described in this example show that rhLAL produced according to methods described herein has desirable pharmacokinetics and pharmacodynamics in vivo.

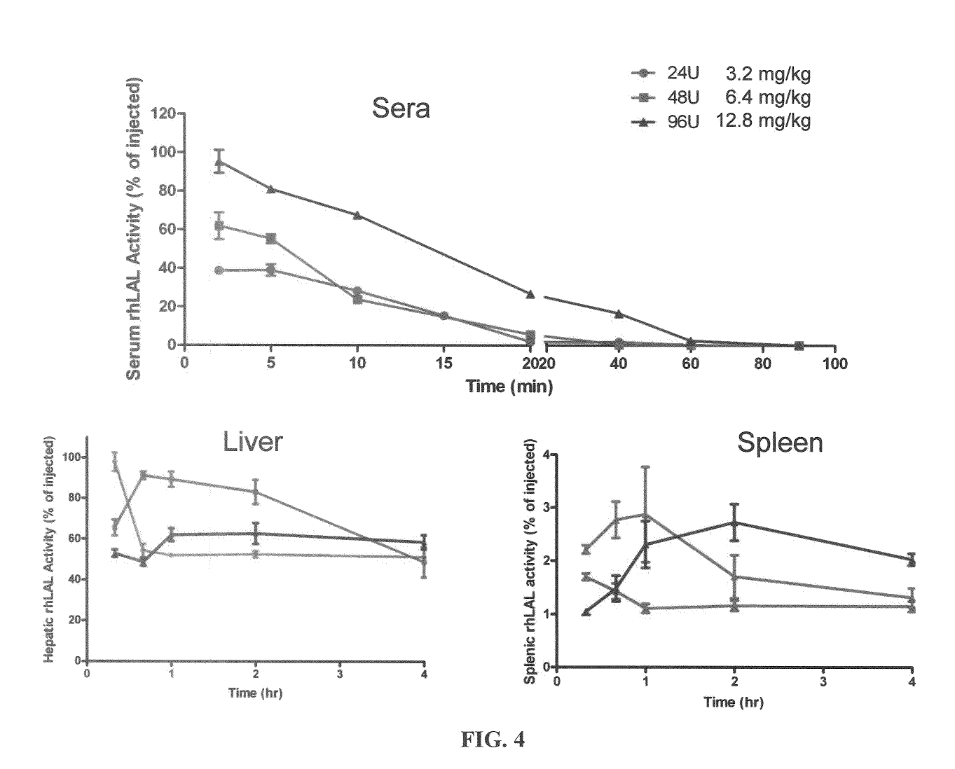

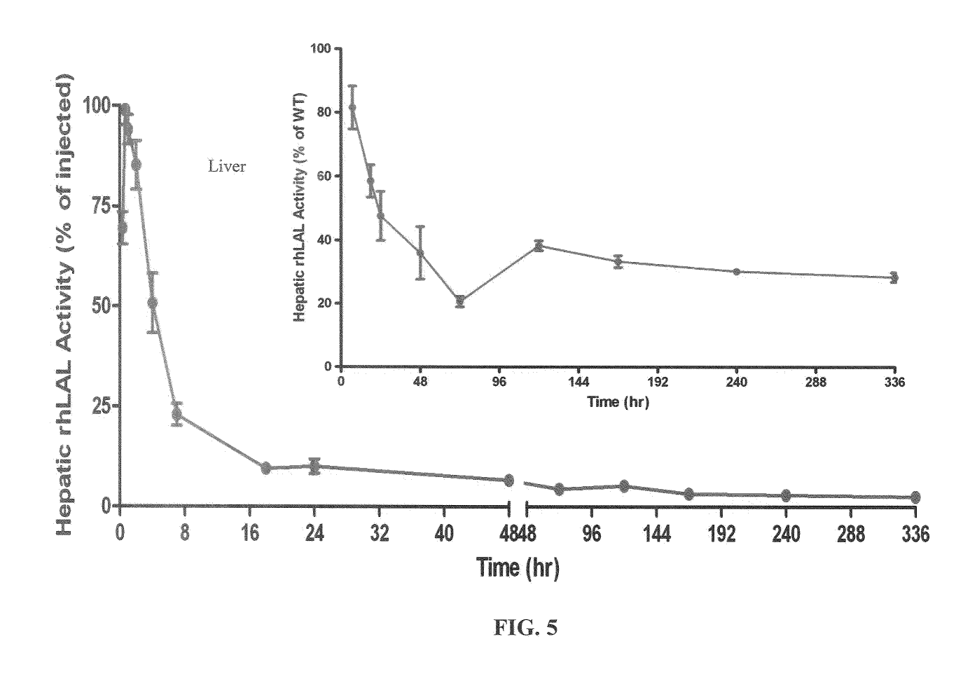

Recombinant human LAL (rhLAL) was expressed and purified as described in Example 1. Three rhLAL doses, 24 U, 48 U and 96 U (3.2, 6.4, and 12.8 mg/kg), were tested in lal^{-/-} mice for pharmacokinetic studies by intravenous administration. Half-life values (t_{1/2}) in sera, liver and spleen were determined for rhLAL at various doses. Half-life values (t_{1/2}) of rhLAL in sera were 10 minutes for 3.2 and 6.4 mg/kg doses and 15 min for 12.8 mg/kg dose (n=5) (FIG. 4, top panel). Surprisingly, half-life values (t_{1/2}) of rhLAL (a 6.4 mg/kg dose) in the liver and the spleen were 5 hours (FIG. 4, lower panels). A time course of liver rhLAL activity in mice injected with a single dose (48 U per mouse) intravenous injection of rhLAL was performed (FIG. 5). The t_{1/2} of both rhLAL enzyme activity and rhLAL protein in serum and in tissues are comparable, suggesting that most of the rhLAL proteins in both serum or tissues are active.

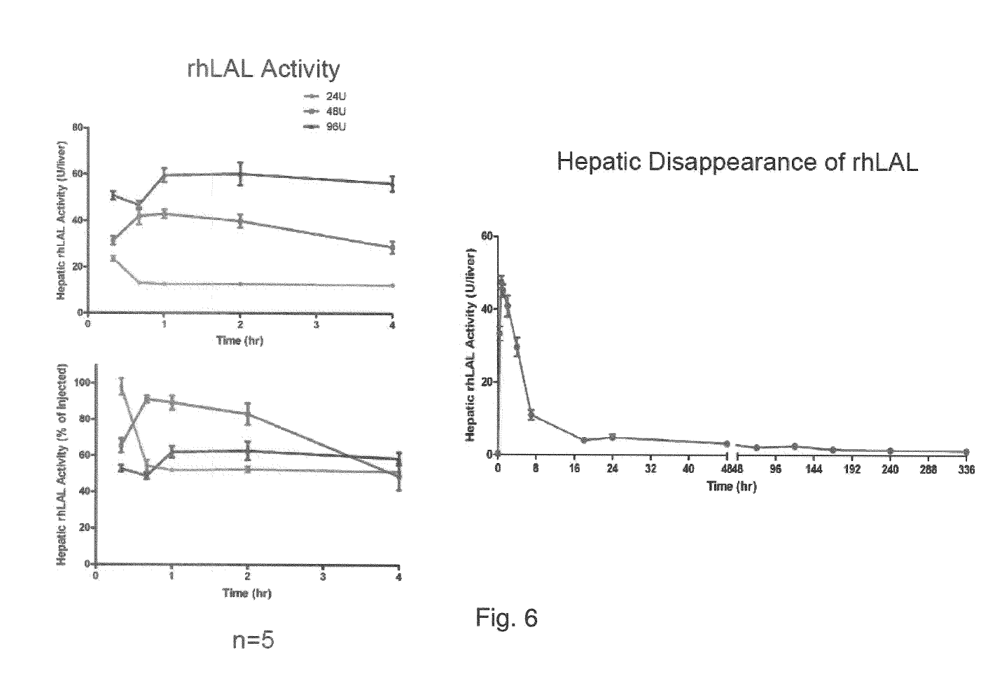

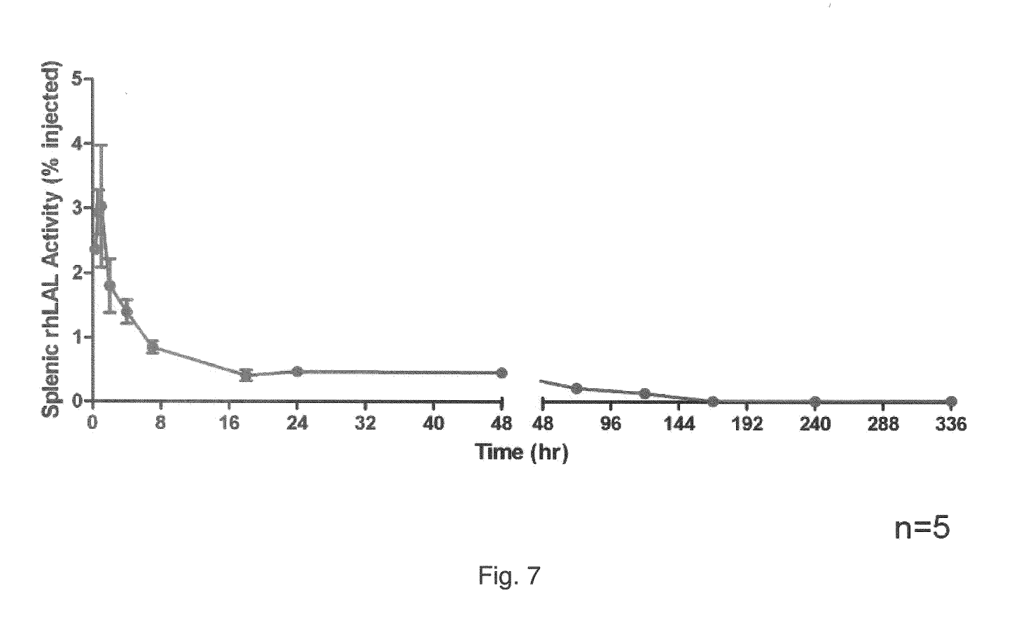

FIGS. 6 and 7 depict further examples of half-life determination of rhLAL in the liver (FIG. 6) and in the spleen (FIG. 7).

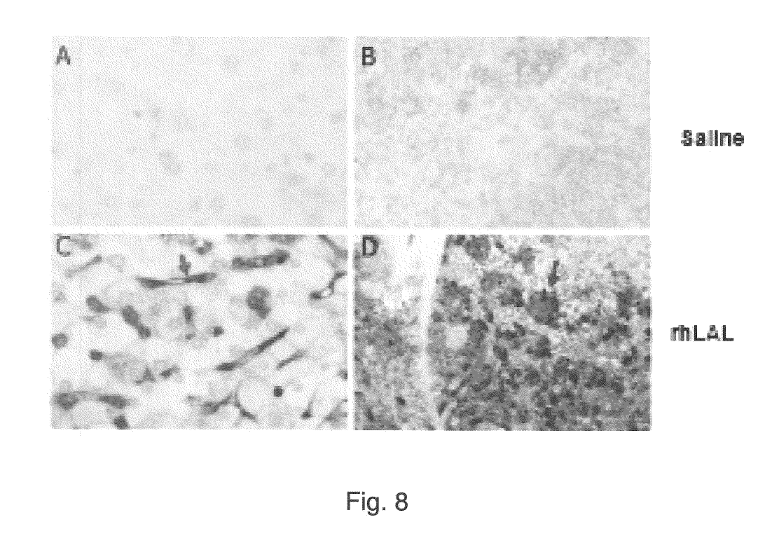

Immunohistochemical analyses of liver and spleen tissues revealed the presence of rhLAL in tissue of injected mice. Paraffin embedded sections of liver and spleen from saline or rhLAL injected lal^{-/-} mice were processed by immunohistochemical staining with anti-hLAL antibody. As shown in FIG. 8, positive signals were evident in the Kupffer cells and in splenic macrophages of a representative section from an lal^{-/-} mouse (arrows). These data demonstrate that both spleen and liver act as cellular targets for rhLAL.

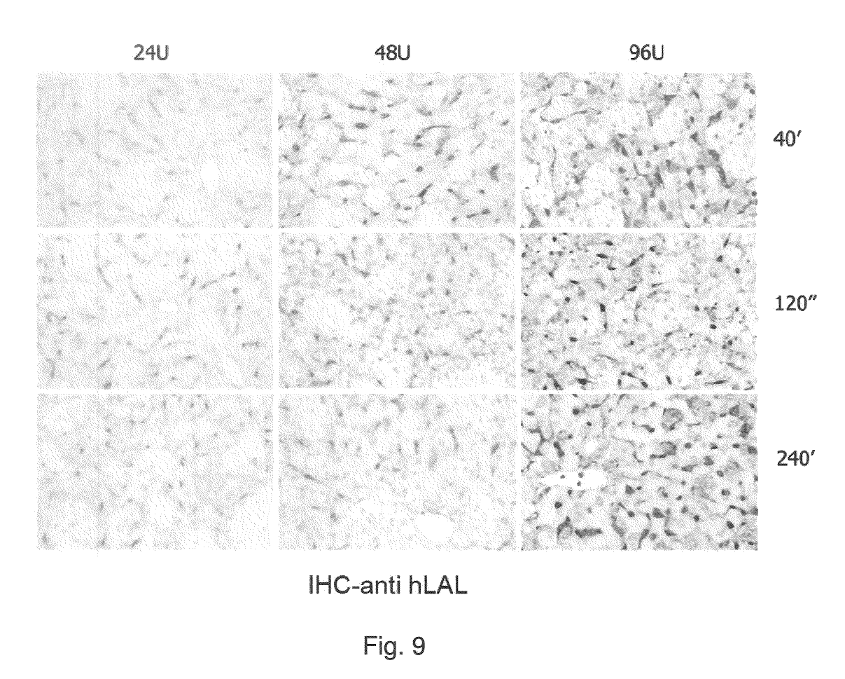

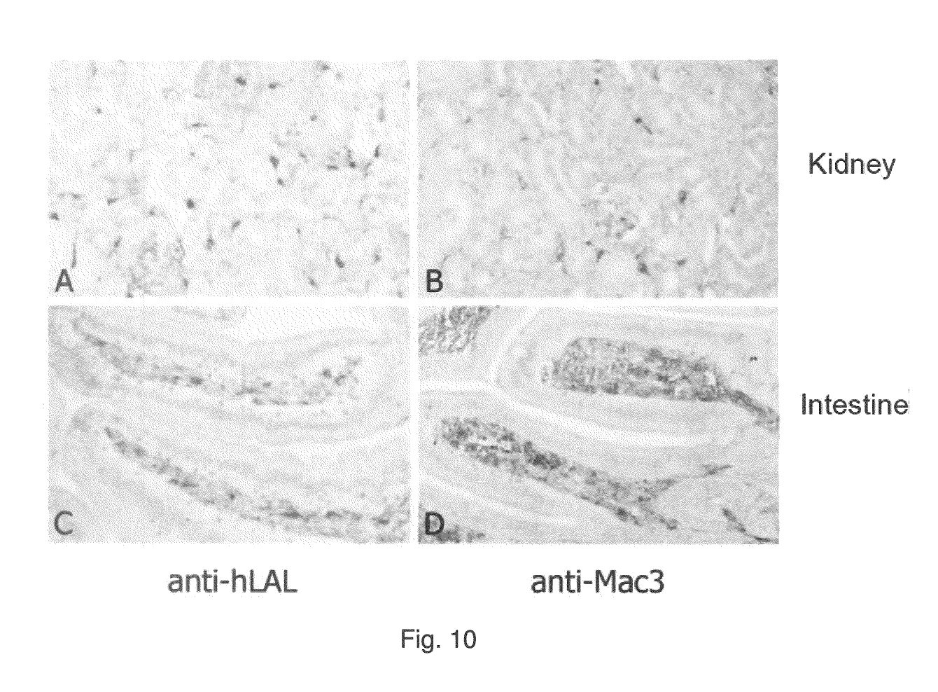

FIG. 9 depicts further examples of rhLAL cellular localization in the liver of lal^{-/-} mice. FIG. 10 shows uptake of rhLAL in macrophages of kidney and intestine (as demonstrated by co-staining with anti-Mac3 antibodies).

### Example 3

### rhLAL Treatment Ameliorates Liver and Spleen Pathology

Experiments described in this example demonstrate that rhLAL can effectively reduce hepatosplenomegaly and lipid accumulation in various tissues. The mouse was used as an animal model.

As described above, lysosomal acid lipase (LAL) hydrolyzes triglycerides (TGs) and cholesteryl esters (CEs), as well as di- and mon-acylglycerols. Lysosomal Acid Lipase Deficiency (LALD) causes either an infantile form known as Wolman disease (iLALD) or a later onset form, known as cholesteryl ester storage disease (CESD or loLALD). The LAL knockout mouse model (lal^{-/-}) resembles human LALD with storage of CEs and TGs in multiple organs, and loss of subcutaneous and omental fat.

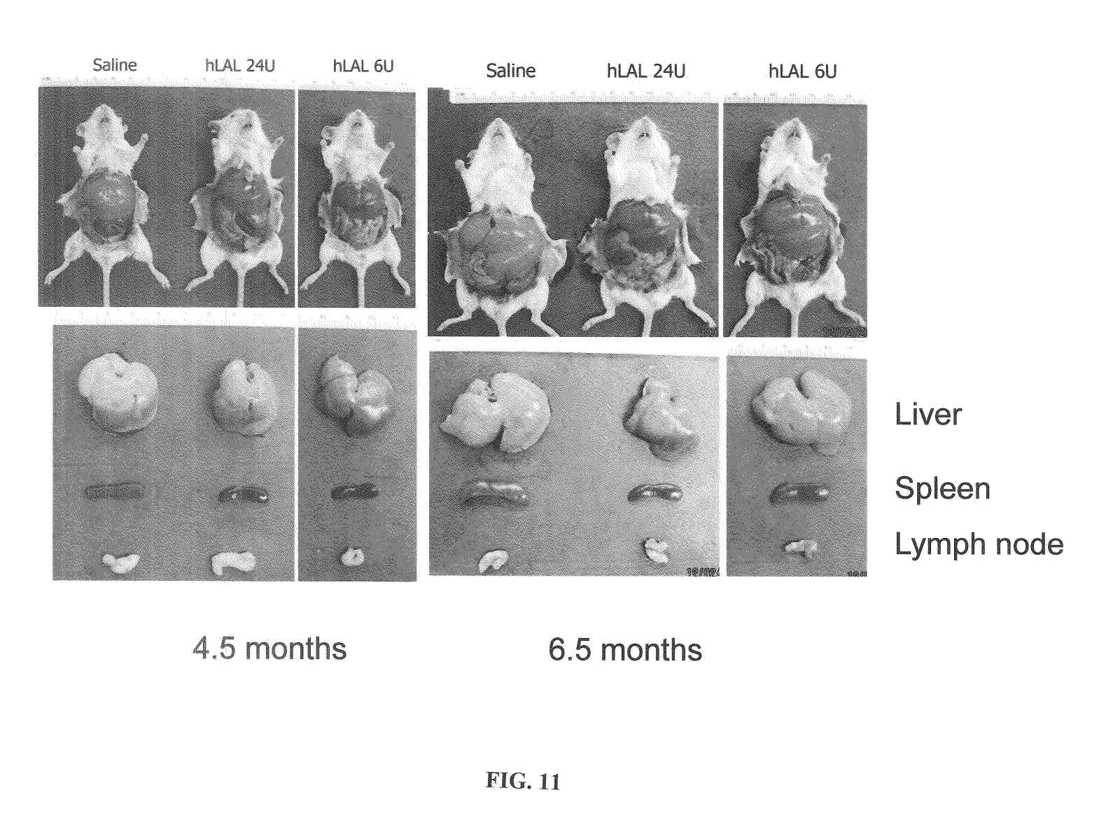

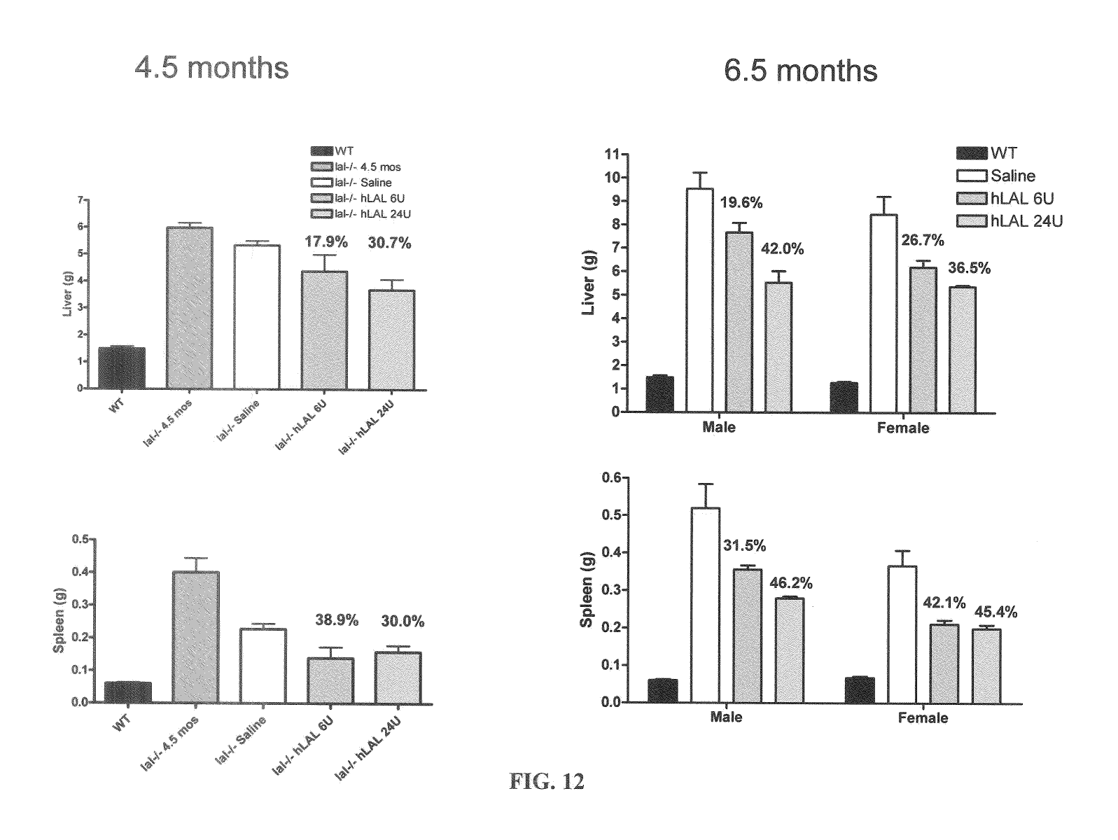

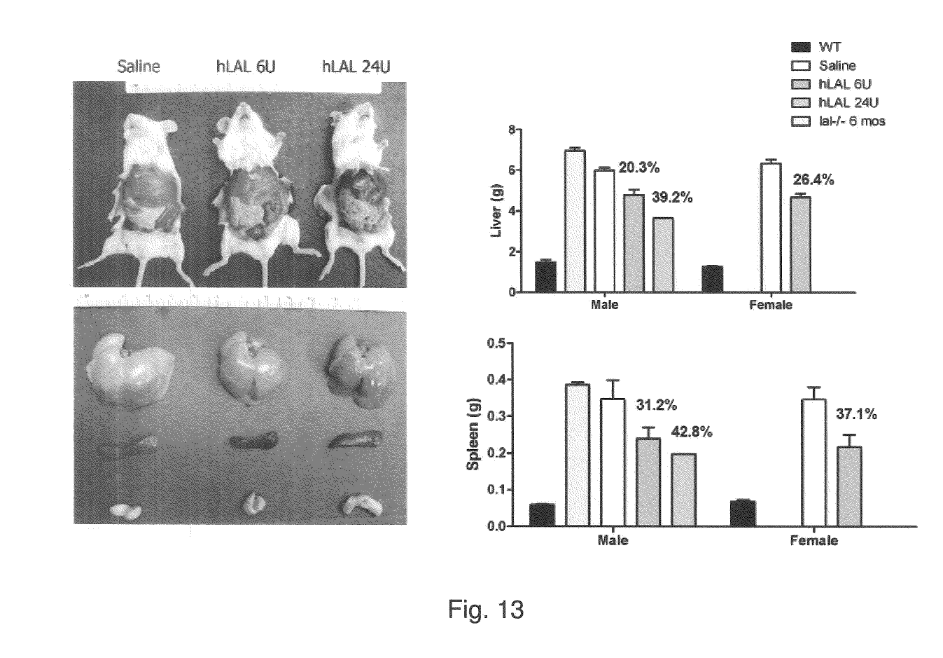

In order to evaluate the effect of rhLAL treatment in liver pathology, animals received two different doses (0.8 mg/kg and 3.2 mg/kg) of rhLAL through tail vein bolus injection weekly for ten weeks in both young (2 months, n=25) and old (4 months, n=17) lal^{-/-} mice for efficacy studies. Gross tissue pathology were assessed and tissue weight at necropsy were determined. Exemplary results are shown in FIGS. 11, 12 and 13. As can be seen in FIG. 11, treatment of mice with weekly injections of rhLAL at a dosage of 0.8 mg/kg and 3.2 mg/kg in both young and old lal^{-/-} mice resulted in resolution of gross liver pathology. As can be seen in FIGS. 12 and 13, treatment of mice with weekly injections of rhLAL at a dosage of 0.8 mg/kg and 3.2 mg/kg in both young and old mice resulted in reduced liver and spleen weight, typically by at least 19% or more as compared to saline-treated animals. Compared to age matched control mice (n=14), intravenous administration of rhLAL reduced liver weight (20-26% with 0.8 mg/kg dose, 39-42.0% for 3.2 mg/kg) and spleen weight (31-42% for 0.8 mg/kg and 36-46.2% for 3.2 mg/kg) in both 2 and 4 month old mice.

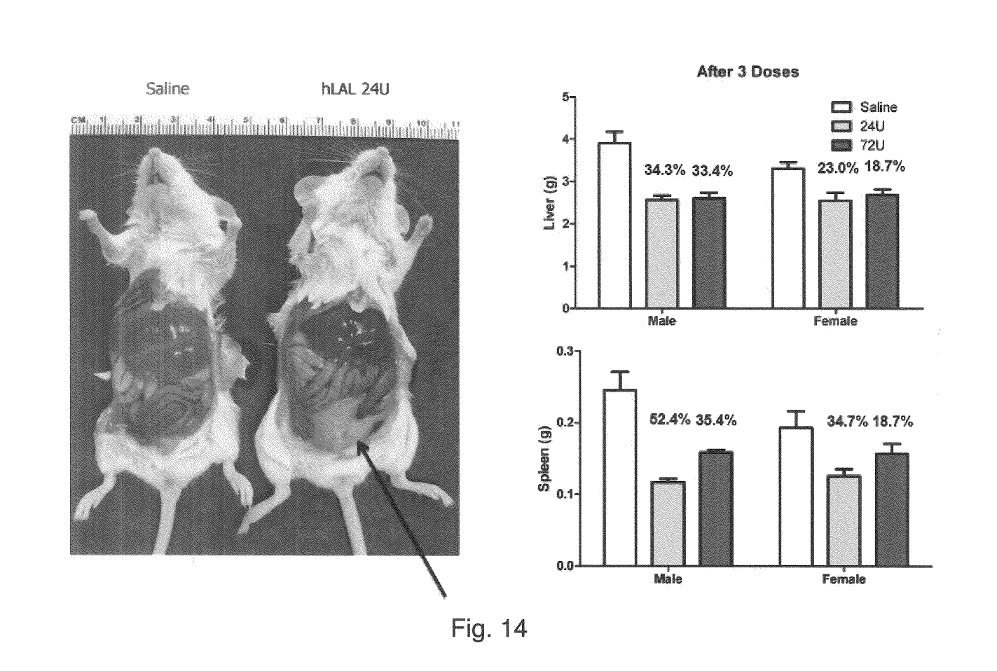

FIG. 14 depicts a further example of improvement of gross liver pathology and reduction of hepatosplenomegaly in lal^{-/-} mice treated rhLAL (24 U or 72 U) as compared to saline-treated animals after 3 doses.

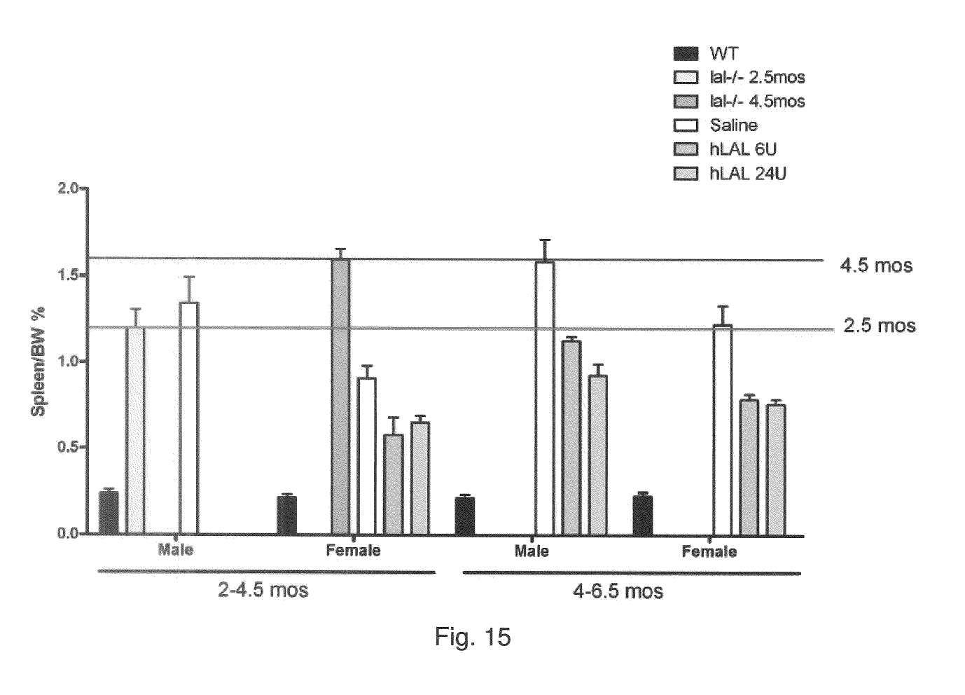

FIG. 15 illustrates exemplary result of treatment of lal−/− mice with rhLAL led to prevention and/or regression of progressive splenomegaly.

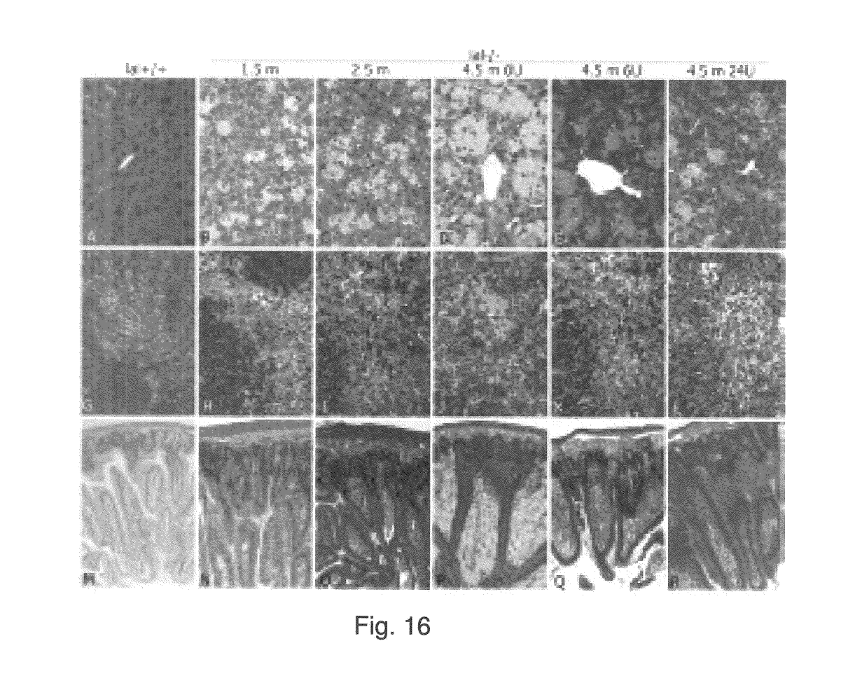

In order to evaluate the correction of lipid storage by rhLAL treatment, hematoxylin and eosin (H & E) staining of tissue was performed. Exemplary results are shown in FIG. 16. Tissues (including liver, spleen, and small intestine) of wild-type and saline- or rhLAL-treated lal^{-/-} mice were examined. As can be seen, correction of lipid storage by rhLAL 6 U was observed in Kupffer cells (panel E), which is similar to a 2.5 month liver section of an untreated lal^{-/-} animal (FIG. 16, panel C). Correction of lipid storage by rhLAL 24 U can also be observed in Kupffer cells (FIG. 16, panel F), which is similar to a 1.5 month liver section of an untreated animal (FIG. 16, panel B). Histological analysis of liver, spleen, small intestine, adrenal gland and lymph node showed significant improvement with both doses, with reversal of phenotype at the highest dose in the 2 month old mice. Taken together, these data demonstrate that rhLAL treatment ameliorates hepatosplenomegaly in lal^{-/-} mice.

rhLAL Treatment Reduces Lipid Accumulation

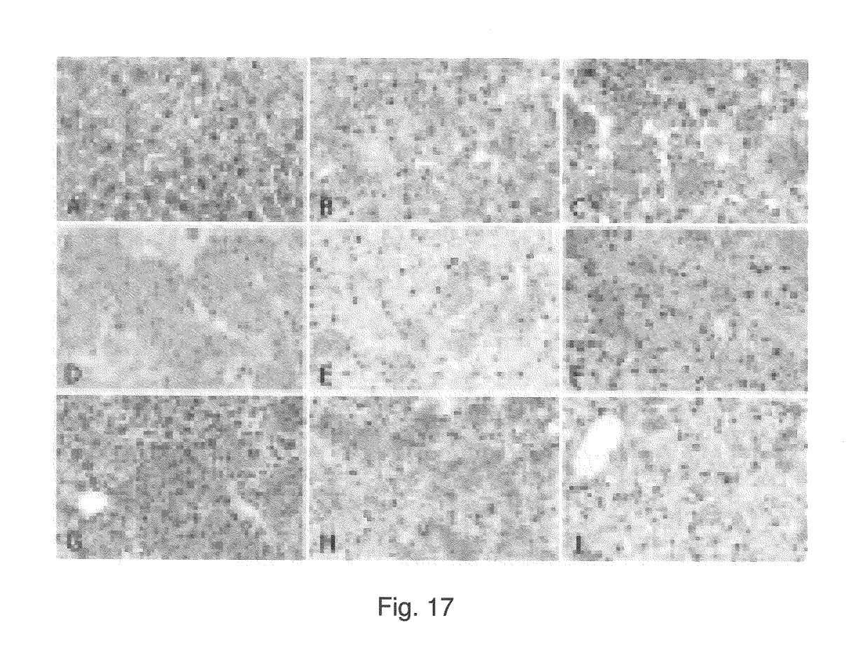

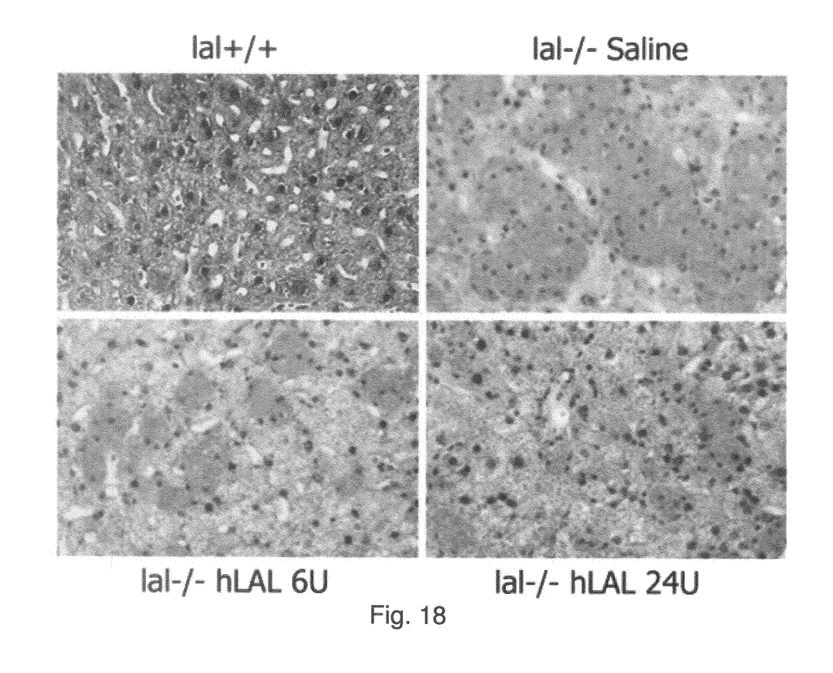

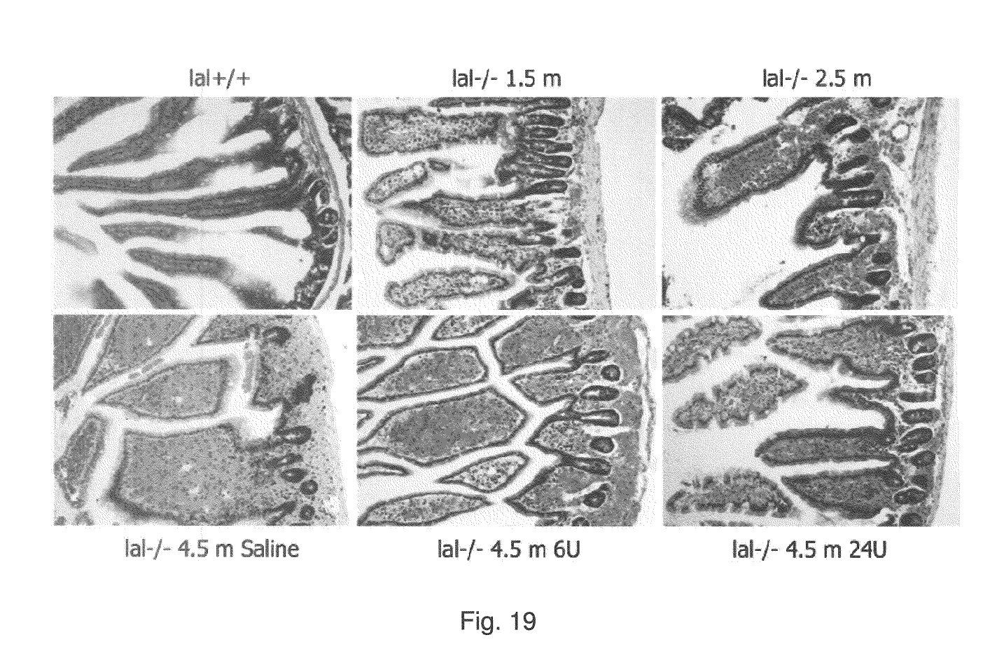

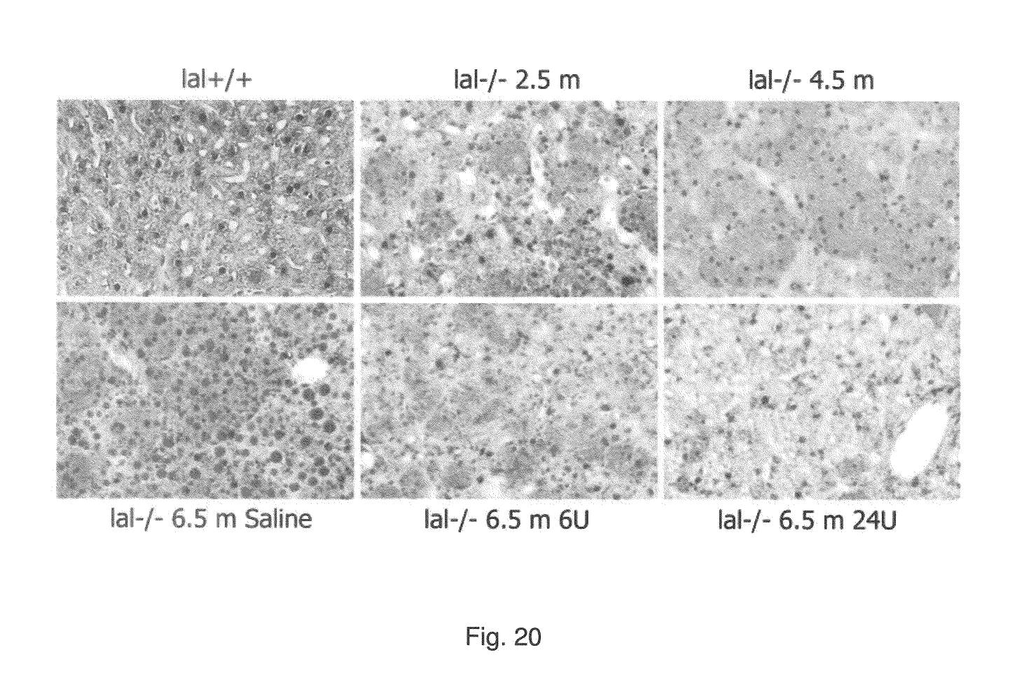

To determine the effect of rhLAL treatment on lipid accumulation, animals received two different doses (0.8 mg/kg and 3.2 mg/kg) of rhLAL through tail vein bolus injection weekly for ten weeks in both young (2 month, n=25) and old (4 month, n=17) lal^{-/-} mice. Exemplary results are shown in FIG. 17. As can be seen, marked reduction of lipid accumulation was found in the livers of treated mice by Oil Red-0 staining. Additional results are shown in FIGS. 18 and 19. FIG. 18 shows young mice treated with rhLAL demonstrated a reduction of neutral lipid in liver by Oil red-O staining. Specifically, mice received two different doses (6 U and 24 U) by intravenous injection weekly from 2 months of age to 4.5 months of age. Reduction of neutral lipid was also seen in small intestine (FIG. 19). Similar results were observed in old mice treated with rhLAL, as demonstrated in FIGS. 20 (liver) and 21 (small intestine) respectively.

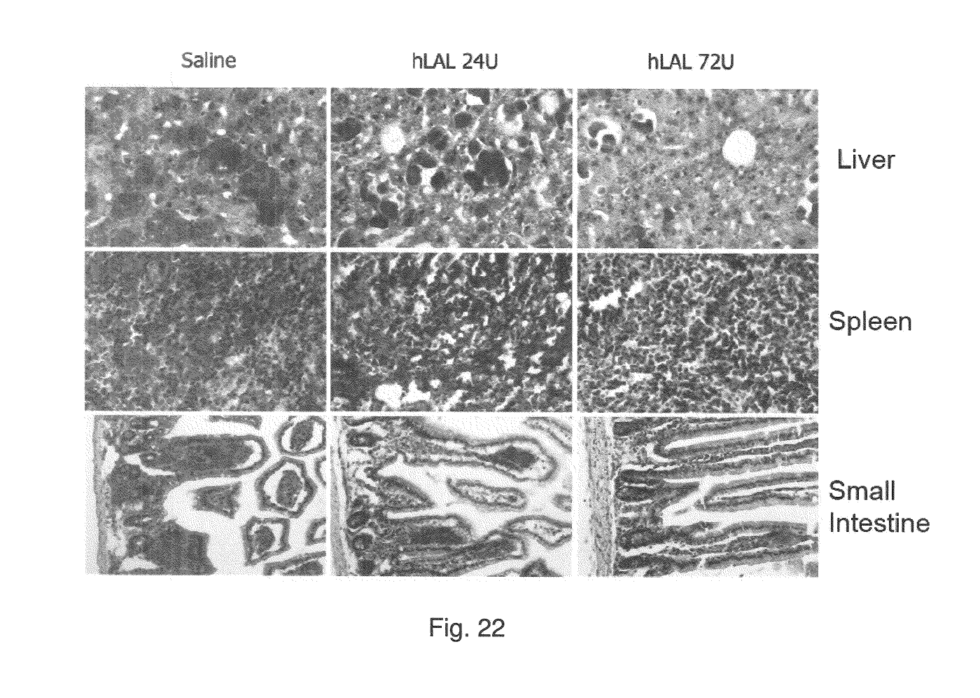

FIG. 22 depicts a further example of reduction of neutral lipids in liver, spleen and small intestine in mice treated rhLAL (24 U or 72 U) as compared to saline-treated animals.

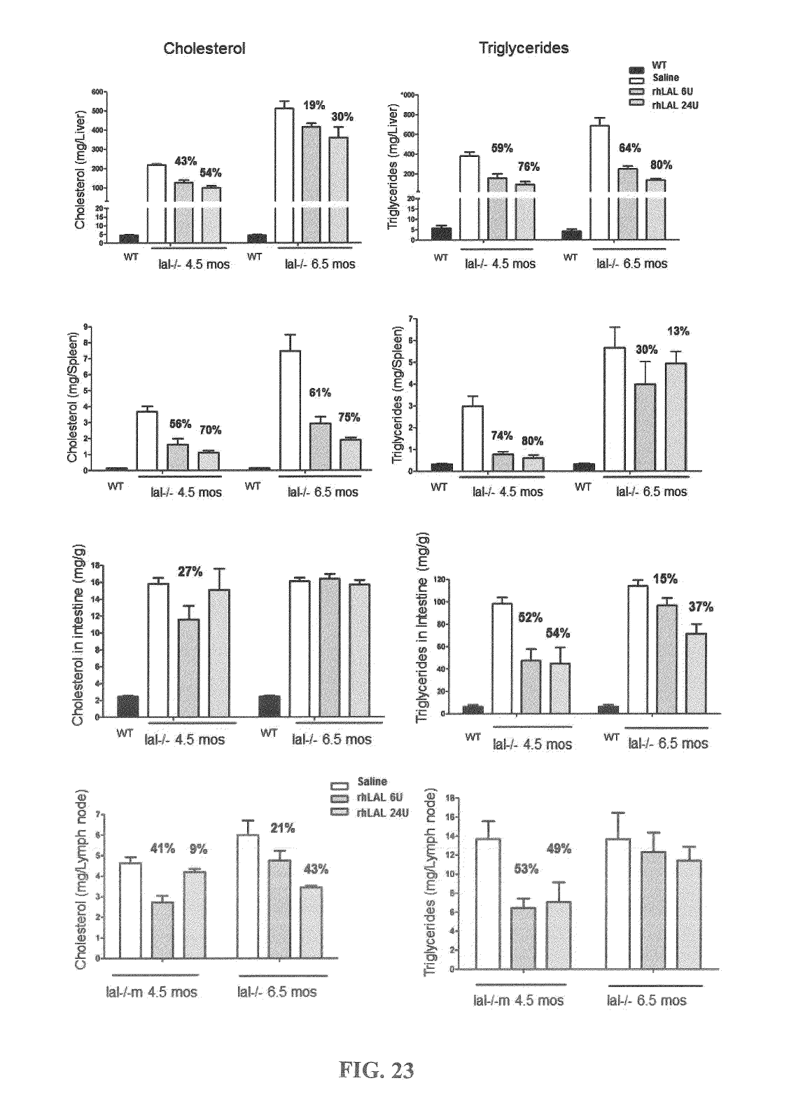

In order to quantitatively measure lipid level reduction in tissues, cholesterol and triglycerides were measured in liver, spleen, intestine, and lymph node of wild-type and saline or rhLAL-treated (hLAL 6 U and hLAL 24 U) lal^{-/-} mice at 4.5 months and 6.5 months of age using lipid analyses methods. As can be seen in FIG. 23, treatment of lal^{-/-} mice with rhLAL led to marked reduction of lipids (e.g., cholesterol and triglycerides) in those tissues.

Taken together, these histochemical and biochemical analyses demonstrated that rhLAL can effectively treat (e.g., ameliorating, inhibiting, delaying onset of, prevent the progression, or cause regression) various systems of LALD disease.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

All publications and patent documents cited in this application are incorporated by reference in their entirety to the same extent as if the contents of each individual publication or patent document were incorporated herein.

1-61. (canceled)

62. A pharmaceutical composition for treating a lysosomal acid lipase deficiency (LALD) disease, comprising a therapeutic effective amount of a recombinant lysosomal acid lipase and a pharmaceutical carrier, wherein the therapeutic effective amount is at least about 0.1 mg/kg body weight and the recombinant lysosomal acid lipase has a half-life of about 5 hours in the liver.

63. The method of claim 62, wherein the lysosomal acid lipase is recombinantly produced from mammalian cells.

64. The method of claim 63, wherein the lysosomal acid lipase is recombinantly produced from human cells.

65. The pharmaceutical composition of claim 62, wherein the LALD disease is Wolman's disease.

66. The pharmaceutical composition of claim 62, wherein the LALD disease is cholesteryl ester storage disease (CESD).

67. The pharmaceutical composition of claim 62, wherein the lysosomal acid lipase has an amino acid sequence at least 80% identical to human lysosomal acid lipase (SEQ ID NO:1).

68. The pharmaceutical composition of claim 67, wherein the lysosomal acid lipase has an amino acid sequence at least 90% identical to human lysosomal acid lipase (SEQ ID NO:1).

69. The pharmaceutical composition of claim 68, wherein the lysosomal acid lipase has an amino acid sequence at least 95% identical to human lysosomal acid lipase (SEQ ID NO:1).

70. The pharmaceutical composition of claim 69, wherein the lysosomal acid lipase is human lysosomal acid lipase (SEQ ID NO:1).

71. A method of treating a lysosomal acid lipase deficiency (LALD) disease, comprising administering to an individual suffering from or susceptible to the LALD disease a therapeutic effective amount of a recombinant lysosomal acid lipase periodically at an administration interval, wherein the therapeutic effective amount is at least about 0.1 mg/kg body weight and the recombinant lysosomal acid lipase has a half-life of about 5 hours in the liver.

72. The method of claim 71, wherein the lysosomal acid lipase is recombinantly produced from mammalian cells.

73. The method of claim 72, wherein the lysosomal acid lipase is recombinantly produced from human cells.

74. The pharmaceutical composition of claim 71, wherein the LALD disease is Wolman's disease.

75. The pharmaceutical composition of claim 71, wherein the LALD disease is cholesteryl ester storage disease (CESD).

76. The pharmaceutical composition claim 71, wherein the lysosomal acid lipase has an amino acid sequence at least 80% identical to human lysosomal acid lipase (SEQ ID NO:1).

77. The pharmaceutical composition claim 76, wherein the lysosomal acid lipase has an amino acid sequence at least 90% identical to human lysosomal acid lipase (SEQ ID NO:1).

78. The pharmaceutical composition of claim 77, wherein the lysosomal acid lipase has an amino acid sequence at least 95% identical to human lysosomal acid lipase (SEQ ID NO:1).

79. The pharmaceutical composition of claim 78, wherein the lysosomal acid lipase is human lysosomal acid lipase (SEQ ID NO:1).


SEQUENCE LISTING (5 sequences)

SEQ ID NO: 1 - protein, 378 aa, Homosapiens
SGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 2 - protein, 21 aa, Homosapien
MKMRFLGLVVCLVLWTLHSEG

SEQ ID NO: 3 - protein, 399 aa, Homosapien
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 4 - protein, 20 aa, Homosapien
DGYILCLNRIPHGRKNHSDK

SEQ ID NO: 5 - protein, 20 aa, Homosapien
MACLEFVPFDVQMCLEFLPS